(19) United States
(12) Patent Application Publication
(10) Pub. No.: US 2023/0293579 A1
(43) Pub. Date: Sep. 21, 2023

(54) CRYSTALLINE ZIRCONIUM PHOSPHATE FOR USE IN THERAPY, PHARMACEUTICAL COMPOSITION, AND USE OF THE SAME

(71) Applicant: CJ Tech Holdings Pte Ltd., Singapore (SG)

(72) Inventor: Christian Gert BLUECHEL, Singapore (SG)

(21) Appl. No.: 18/006,164

(22) PCT Filed: Jul. 21, 2021
(86) PCT No.: PCT/SG2021/050429

§ 371 Date: Jan. 20, 2023

(30) Foreign Application Priority Data
Jul. 20, 2020 (SG) 10202006928V

(51) Int. Cl. A61K 33/42; A61K 9/00

(52) U.S. Cl. CPC A61K 33/42; A61K 9/0053

(57) ABSTRACT

The present disclosure provides a crystalline zirconium phosphate and a pharmaceutical composition comprising crystalline zirconium phosphate and optionally one or more therapeutically effective agents for use in therapy. There is also provided a use of crystalline zirconium phosphate in the manufacture of a pharmaceutical composition for the treatment of a patient with abnormally high levels of one or more toxins.

### CROSS-REFERENCE TO RELATED APPLICATION

This application claims the benefit of priority of Singapore Patent Application No. 10202006928V, filed 20 Jul. 2020, the contents of it being hereby incorporated by reference in its entirety for all purposes.

### TECHNICAL FIELD

Various aspects of this disclosure relate to a medical use of zirconium phosphate in a crystalline modification.

### BACKGROUND

In prevention or therapy for kidney and/or liver dysfunction it is necessary to facilitate removal of toxins from the body. The toxins can be cationic salts such as potassium, sodium and ammonium salts, or the toxins can be organic toxins such as urea, creatinine, uric acid, hippurates and homocysteine to name just a few. Dialysis is used to remove many of these toxins. Dialysis treatments severely limit a patient's core life functions. Furthermore, dialysis alone may not lower the concentration of serum toxins to acceptable levels. It would therefore be desirable to provide other means to lower the concentration of serum toxins to acceptable levels, which may support patients already on dialysis, but also to prevent or delay patients from requiring dialysis, i.e. pre-dialysis treatment. Hence, it may be desirable to reduce the necessary dosages of dialysis, or, by carrying out pre-dialysis treatment, to preserve kidney function such that the time before dialysis is started is delayed, resulting in an improved quality of life of the patient.

The normal plasma potassium level is typically maintained at about 3.5 to 5 mEq/L, and toxicity can begin at levels of over 6 mEq/L. Excess potassium (hyperkalemia) can occur in patients with chronic renal dysfunction or failure. It can also occur in patients on treatment regiments including three-times-per-week dialysis, especially on weekends when the interval between dialysis treatment is longer.

Current therapy for patients with hyperkalemia includes either dialysis and/or oral intake of 15 to 30 grams kayexelate, which contains polystyrene sulfonate sodium (PSS), several times per day until the potassium level decreases or dialysis can be implemented to remove potassium. The PSS is used to adsorb potassium from the intestine and colon. While the kidneys are the main excretory routes for potassium, it is well known that the concentration of potassium is much higher in the colon than in the small intestine or in the blood. Furthermore, the colon mucosa has an active transport pump for potassium that transports potassium from the peritoneal into the colon. This is similar to the potassium pump that exists in the distal tubule of the kidney. Unfortunately, it has been observed that PSS also exhibits a greater affinity for divalent cations, such as calcium and magnesium than for mono-valent cations. Consequently much of the capacity of the PSS for adsorbing cations is already exhausted by the time the PSS reaches the colon. This obviously reduces the effectiveness of administering PSS to treat hyperkalemia.

Additionally, PSS can aggregate into a solid mass and cause an obstruction within the intestines, intestinal ischemia and ulcers. These risks are increased in patients with diminished gut activity or constipation, which is symptomatic of patients with kidney dysfunction. To offset the risks of such obstruction and to increase potassium removal by the gut, PSS treatment is given in combination with a non-absorbable sugar, such as sorbitol in an amount that assures the development of diarrhea in the patient. It is not surprising that many patients complain of the volume of sorbitol needed, its sweet taste, and obviously, the diarrhea and accompanying abdominal discomfort.

Typically the blood urea level is also high with kidney dysfunction. The kidney is a principle organ for urea excretion and the liver is the only source of urea production. The balance of urea formation by the liver and kidney excretion normally maintains a blood urea nitrogen (BUN) level of about 13 to 20 mg per deciliter (mg %). Because of kidney dysfunction, the BUN level can range from about 50 to about 200 mg % depending upon the daily protein intake and/or degree of protein breakdown in the body. Urea is produced in the liver by transfer of nitrogen from amino acids, ammonium and other nitrogenous chemicals, and the urine excretion of urea is the major method of the body to remove excess nitrogen. Frequently, patients with decreased kidney capacity are forced to eat a diet with strict limitations in protein in an effort to lower their BUN level. Approximately 25% of the urea produced every day passes into the gut, where bacteria with the urease enzyme break it down into ammonium and carbonate. Most of the ammonium is absorbed by the gut and converted in the liver back into urea. Excess urea and ammonium in the gut can cause uremia (wide-ranging symptoms of kidney failure). However, removal of a significant amount of ammonium from the gut would significantly lower BUN levels and reduce the onset of uremia.

In addition, patients with liver dysfunction and/or failure also exhibit an elevation of blood level ammonium. Elevated ammonium has a rough correlation to brain dysfunction and liver failure encephalopathy (brain dysfunction). The standard therapy for hyperammonemia is the use of non-absorbable saccharide such as lactulose. However, this therapy is not very effective for decreasing blood level ammonium.

Current oral sorbents under investigation are disclosed, for example, in US 2019/0008894 A1. Therein, an oral sorbent therapy is provided using a cation exchanger that could be either hydrogen-loaded polystyrene sulfonate (H-PS) or hydrogen-loaded zirconium phosphate (H-ZP) and an anion exchanger that could be hydroxy-loaded zirconium oxide (or hydrous zirconium oxide, collectively abbreviated as OH-ZO). The H-ZP utilized in this system is non-crystalline. However, such a system has problems with regard to selectivity for monovalent ions. This results in a limited capacity for removal of monovalent ions, such as potassium and ammonium, over divalent ions, such as calcium and magnesium, since the binding capacity of the oral sorbent would be used up with divalent ions. This is because divalent ions are preferentially bound which triggers release of monovalent ions even after they are bound. Additionally, this oral sorbent does not sufficiently remove sodium, or even releases sodium, which results in the sodium level still being considerably high in the gut.

US 2019/0008894 A1 further discloses crystalline zirconium cyclosilicate (ZS9) for use as an oral sorbent. However, commonly encountered disadvantages with this oral sorbent ZS9 are a partial solubility, resulting in toxicity problems due to the silicates being absorbed by the digestive tract. Ideally, a sorbent would have to be completely insoluble in order to avoid toxicity. Additionally, ZS9 has disadvantages with regard to pharmacological suitability, since, for example, there are concerns relating to the particle size and inability for granulation of the compound, relating in toxicity concerns with nano- and micrometer sized particles. As for the above combination of H-PS or H-ZP and OH-ZO, ZS9 has a limited capacity for removing monovalent ions, such as potassium and ammonium. Most importantly, this oral sorbent does not sufficiently remove sodium, which results in the sodium level still being considerably high in the gut. Furthermore, the process of manufacturing ZS9 is very complicated and time-consuming, and therefore costly.

Therefore, there is a need to solve, or at least ameliorate, the above mentioned problems.

### SUMMARY

In a first aspect, there is provided crystalline zirconium phosphate for use in therapy.

In a second aspect, there is provided a pharmaceutical composition comprising crystalline zirconium phosphate and optionally one or more therapeutically effective agents.

In a third aspect, there is provided a use of crystalline zirconium phosphate in the manufacture of a pharmaceutical composition for the treatment of a patient with abnormally high levels of one or more toxins.

In a fourth aspect, there is provided a method of treating animals, said method comprising: selecting an animal capable of deriving a benefit from lower levels of one or more serum toxins, comprising administering to said animal a pharmaceutical comprising a crystalline zirconium phosphate sorbent.

### DETAILED DESCRIPTION

In general, the present disclosure relates to a crystalline zirconium phosphate for use in therapy. The crystalline zirconium phosphate can function as a cation exchanger and exchange one or more cations and adsorb cationic toxics, such as, ammonium cations, potassium cations, sodium cations, calcium cations, magnesium cations, from a patient. In other words, the crystalline zirconium phosphate functions as a sorbent, and in some embodiments, as an oral sorbent. Hence, in various embodiments, there is provided a crystalline zirconium phosphate for use in therapy for treating animals. The therapy involves selecting an animal capable of deriving a benefit from lower levels of one or more serum toxins. The present disclosure relates to pharmaceutical compositions, medicaments and methods of treating patients having elevated levels of one or more serum toxins including, but not restricted to, patients with liver and renal dysfunction. Hence, the present disclosure finds particularly advantageous use for patients suffering from early onset to end-stage renal and/or liver dysfunction. While the actual nature of the disclosure covered herein can only be determined with reference to the claims appended hereto, certain forms and features, which are characteristic of the preferred embodiment disclosed herein, are described briefly as follows.

Advantageously, crystalline zirconium phosphate, in particular γ (gamma)-zirconium phosphate, has improved properties that allow for use as a sorbent, particularly as an oral sorbent. Such improved properties, for example in view of currently known ZS9 sorbents, include the complete insolubility of crystalline zirconium phosphate, which results in decreased toxicity. Additionally, crystalline zirconium phosphate, in particular γ (gamma)-zirconium phosphate, is suitable to be granulated such that it may be in a more efficient form for oral sorption and may be better manufactured into a pharmaceutical composition. Furthermore, the hydrogen form of crystalline zirconium phosphate does not release sodium, but may provide a net sodium removal. More advantageously, there is a strong binding of NH₃ over NH₄⁺, resulting in more efficient binding of NH₃ in the gut. Finally, binding of potassium is close to irreversible for crystalline zirconium phosphate.

Improved properties, for example contrasted with amorphous zirconium phosphate, include a high selectivity for monovalent cations, whereas only a low binding has been observed for calcium and magnesium. Furthermore, since crystalline zirconium phosphate has a high capacity for removal of sodium, there is less release of sodium, but more efficient sodium removal. Without being bound to theory, it is believed that these advantages are achieved by the size of the crystal lattice of crystalline zirconium phosphate, which is ideal for monovalent cations and NH₃ but not for divalent cations. Hence, the crystalline zirconium phosphate of the present disclosure has a higher binding capacity for potassium (K) and NH₃ than cyclosilicate and amorphous zirconium phosphate. Moreover, there is a large extent of internal binding relative to surface binding in the crystalline zirconium phosphate. Then, the solubility of pure crystalline forms is lower than those of amorphous forms, which helps to reduce solubility in the digestive tract. In fact, crystalline forms are essentially insoluble in the digestive tract. More advantageously, the crystal lattice favours binding of NH₃ over NH₄⁺. A particular advantage of the crystalline form is that it can be produced completely Na-free.

Moreover, the crystalline zirconium phosphate can be used without causing bowel obstruction or bowel irritation. Thus, the crystalline zirconium phosphate in accordance with the present disclosure may be administered without need to induce diarrhea by administering non-absorbable saccharides. This can avoid patient complaints about the bad taste, nausea and abdominal pain created by the non-absorbable saccharides.

In the crystalline zirconium phosphate of the present disclosure, more than 50wt %, optionally more than 70wt %, optionally more than 90wt %, optionally more than 95wt %, optionally more than 99wt % of the crystalline zirconium phosphate may be present as α-zirconium phosphate.

In the crystalline zirconium phosphate of the present disclosure, more than 50wt %, optionally more than 70wt %, optionally more than 90wt %, optionally more than 95wt %, optionally more than 99wt % of the crystalline zirconium phosphate may be present as γ-zirconium phosphate.

In the crystalline zirconium phosphate of the present disclosure, the crystalline zirconium phosphate may be present as a mixture of α-zirconium phosphate and γ-zirconium phosphate.

There are various applications that are possible for crystalline zirconium phosphate (or similar monovalent-selective cation exchangers) as sorbent, particularly as an oral sorbent. Depending upon the disease state being treated, the crystalline zirconium phosphate could be loaded with differing cations. The crystalline zirconium phosphate sorbent can be loaded with any number of monovalent cations and still remove ammonium. For treatment of hyperkalemia in patients with some kidney function, sodium loading would be advantageous since the absorbed sodium would increase urine flow and by this increase kidney excretion of potassium. For treatment of chronic kidney insufficiency to remove urea and potassium, sodium release would only add to the sodium and fluid overload of the patients. The crystalline zirconium phosphate loading with hydrogen would be advantageous. The hydrogen released would balance an increase in pH by the generation of carbonate from urease within the gut bacteria.

The crystalline zirconium phosphate may have one or more adsorbed, exchangeable cations selected from hydronium cations, calcium cations, sodium cations, potassium cations, magnesium cations and mixtures thereof. Preferably, the crystalline zirconium phosphate may be selected to selectively release or desorb cations such as calcium and hydronium into the patient and adsorb ammonium and potassium cations from the patient. Examples of intestinal permeability enhancing agents at low concentrations include alcohol, polyethylene glycol, glycerin, propylene glycol, acetone, and polyvinyl alcohol. The pharmaceutical composition can be administered orally, through an ostomy inlet, or rectally.

The composition comprises a monovalent cation exchanger that comprises a crystalline zirconium phosphate sorbent. The crystalline zirconium phosphate may have exchangeable cations absorbed thereon. The exchangeable cations can be selected to include proton, hydronium, sodium, potassium, calcium, magnesium and mixtures thereof.

The crystalline zirconium phosphate sorbent can be selected to include a wide variety of synthetic and natural cation or anion exchangers. In preferred embodiments, the cation exchangers can have a wide variety of sizes, shapes and ionic charges to allow them to effectively bind monovalent cations, minimizing the competitive binding or adsorption by di- and tri-valent cations. Specific examples of the preferred cation exchangers can be found and are described in U.S. Pat. No. 6,099,737 A entitled “Process for Removing Toxins from Blood Using Zirconium Metallate or a Titanium Metallate Composition”; and U.S. Pat. No. 6,332,985 B1 entitled “Process for Removing Toxins from Bodily Fluids Using Zirconium or Titanium Microporous Compositions”, all of which are incorporated by reference in their entirety. Specific examples of the preferred anion exchangers can be found and are described in US 2019/0008894 A1. In this patent application, the anion exchanger may be OH-ZO. Anions such as phosphate, sulfate, and chloride are bound by the anion exchanger, releasing hydroxide.

The crystalline zirconium phosphate for use in the present disclosure may be an acidic, inorganic cation exchange material and have a layered structure with formula (I) or (II):

Zr(HPO₄)₂nH₂O (I), or

(Zr(PO₄)(H₂PO₄)nH₂O) (II).

The crystalline zirconium phosphate of this disclosure may have spaces for (alkali) metals within the framework. These (alkali) metals are described as exchangeable cations meaning that they can be exchanged for other (secondary) cations. The exchangeable cations can be exchanged for other (alkali) metal cations (K+, Na+, Rb+, Cs+), alkaline earth cations (Mg²⁺, Ca²⁺, Sr²⁺, Ba²⁺), hydronium cation (H₃O⁺), ammonium cations (NH₄⁺), or mixtures thereof. The methods used to exchange one cation for another involve contacting the sorbent with a solution containing the desired cation at exchange conditions. The crystalline zirconium phosphate may be suspended in an aqueous solution that also includes an excess of the cations to be exchanged for the cations that are already contained in the sorbent. Typically the desired cations are provided in about 2 to about 10 molar excess based upon the number of moles of cations (or available valences on the sorbent) already absorbed on the sorbent. The aqueous solution can be maintained at a temperature of between about 25° C. to about 100° C. and for a time of about 10 minutes to about 2 hours to allow the exchange reaction to go to completion. Thereafter the suspension is filtered to collect and retain the crystalline zirconium phosphate, which may be dried either at room temperature or under elevated temperatures, preferably not greater than about 50° C., or greater than about 150° C.

The crystalline zirconium phosphate of this disclosure may have high thermal and chemical stability, solid state ion conductivity, resistance to ionizing radiation, and the capacity to incorporate different types of molecules with different sizes between their layers. There are various crystalline phases of zirconium phosphate which vary in their interlaminar spaces and their crystalline structure. The crystalline zirconium phosphate phases may, for example, be the alpha (α-) (Zr(HPO₄)₂·H₂O) and the gamma (γ-) (Zr(PO₄)(H₂PO₄)₂H₂O) phase. The layered structure of α-zirconium phosphate may consist of Zr(IV) ions situated alternately slightly above and below the ab plane, forming an octahedron with the oxygen atoms of the tetrahedral phosphate groups. Of the four oxygen atoms in the phosphate groups, three are bonded to three different Zr atoms, forming a cross-linked covalent network. The fourth oxygen atom of the phosphate is perpendicular to the layer and pointed toward the interlayer region. In the interlayer region is localized a zeolitic cavity where a basal water molecule resides, forming a hydrogen bond with the OH group of the phosphate that is perpendicular to the layer. The a phase of zirconium phosphate is under the P21/n space group, with typical cell dimensions of a=9.060 Å, b=5.297 Å, c=15.414 Å, α=γ=90°, β=101.71° and Z=4.21. The basal interlayer distance for the α-zirconium phosphate is 7.6 Å, where 6.6 Å is the layer thickness. The distance between adjacent orthophosphates on one side of the layer is 5.3 Å. There are two phosphates in each ab plane in the surface layer forming a “free area” of 24 Å² associated to each phosphate group. Variations in the cell dimensions may occur dependent on the state of the material.

The structure of γ-Zirconium phosphate may comprise Zr(IV) atoms octahedrally coordinated to four different oxygen atoms of an orthophosphate. The other two resting octahedral position of the Zr(IV) atoms may be occupied by two different dihydrogen phosphate groups. The orthophosphate molecules are located alternatively above and below the ab main plane and the dihydrogen phosphates are in the layer edges crosslinked by two of their oxygen atoms to two different zirconium atoms. The remaining two hydroxyl groups of the dihydrogen phosphate are pointing toward the interlayer gallery forming a pocket where a hydrogen bond is formed with the interlayer water molecules. The basal interlayer distance for γ-Zirconium phosphate is 12.2 Å, and the area surrounding the dihydrogen phosphate on the surface of the layers is 35 Å².

This framework of the crystalline phases of zirconium phosphate may result in a structure having an intracrystalline pore system with uniform pore diameters, i.e., the pore sizes are crystallographically regular. Advantageously, the sorbents of this disclosure may be capable of selective ion exchange of ammonium cations and/or potassium cations. This is hypothetically rationalized with the unique crystal structure of crystalline zirconium phosphate.

One specific composition for use in the present disclosure may include a crystalline zirconium phosphate that may include a combination of calcium and hydronium ions absorbed thereon. The calcium and hydronium ions may be absorbed on the crystalline zirconium phosphate sorbent in a ratio (Ca²⁺:H₃O⁺) of between about 0.1 and about 0.9, more preferably in a ratio between about 0.4 and about 0.6. One specific composition for use in the present disclosure may include a crystalline zirconium phosphate that may include a combination of sodium and hydronium ions absorbed thereon. The sodium and hydronium ions may be absorbed on the crystalline zirconium phosphate sorbent in a ratio (Na⁺:H₃O⁺) of between about 0.1 and about 0.9, more preferably in a ratio between about 0.4 and about 0.6. In specific embodiments, the crystalline zirconium phosphate may be provided to have a cation capacity from between about 0.3 mEq per gram of sorbent to about 1.5 mEq per gram of sorbent. Alternatively, the crystalline zirconium phosphate may be substantially or completely sodium free. “Substantially” means, in this context, that a composition comprising the crystalline zirconium phosphate includes less than 5 wt % of sodium, preferably less than 1 wt % of sodium, preferably less than 0.5 wt % of sodium, or preferably less than 0.1 wt % of sodium.

The crystalline zirconium phosphate of the present disclosure can be provided as a solid in a wide range of particle sizes depending upon the desired mode of administration and co-additives. In some embodiments, the crystalline zirconium phosphate of the present disclosure may be provided with an average particle size of between about 1 to about 500 micrometers, or of between about 1 to about 150 micrometers, more preferably between about 3 to about 50 micrometers, 5 and about 20 micrometers, more preferably between about 5 and about 10 micrometers. The crystalline zirconium phosphate of the present disclosure may be provided as a fine powder. The particle size, as referred to herein, is obtained by using the sieve analysis known to the person skilled in the art, for example, using U.S. Standard Sieve according to ASTM E11-20.

In preferred embodiments, the crystalline zirconium phosphate of the present disclosure may be provided as granules. The granules may have a particle size of between about 1 to about 150 micrometers, of between about 150 micrometers to about 500 micrometers, or of between about 200 micrometers to about 400 micrometers, or of between about 250 micrometers to about 350 micrometers, or of between about 200 micrometers to about 300 micrometers.

The crystalline zirconium phosphate may be used in the treatment of a patient with abnormally high levels of one or more toxins, and/or in the prevention or treatment of renal and hepatic insufficiency, and/or in the prevention or treatment of kidney failure.

In another aspect, there is provided a pharmaceutical composition comprising the crystalline zirconium phosphate.

In the pharmaceutical composition of the present disclosure, more than 50wt %, optionally more than 70wt %, optionally more than 90wt %, optionally more than 95wt %, optionally more than 99wt % of the crystalline zirconium phosphate may be present as α-zirconium phosphate.

In the pharmaceutical composition of the present disclosure, more than 50wt %, optionally more than 70wt %, optionally more than 90wt %, optionally more than 95wt %, optionally more than 99wt % of the crystalline zirconium phosphate may be present as γ-zirconium phosphate.

The crystalline zirconium phosphate may be present as a mixture of α-zirconium phosphate and γ-zirconium phosphate in the pharmaceutical composition.

The pharmaceutical composition may comprise one or more therapeutically effective agents. The one or more therapeutically effective agents may include a carbon agent, one or more anion exchangers, zinc oxide, water absorbers, intestinal tissue permeability-enhancing agents, diuretic, and/or a coating.

The pharmaceutical composition may comprise one or more anion exchangers. The pharmaceutical composition can include anion exchangers, and in particular phosphate binders, for example, hydroxy-loaded zirconium oxide.

The pharmaceutical composition may comprise zinc oxide. The zinc oxide can be combined with the crystalline zirconium phosphate in the pharmaceutical composition of the present disclosure, which is then administered to the patient. Alternatively, the zinc oxide can be administered to the patient separately from the crystalline zirconium phosphate either through the same or a different administration route. Advantageously, zinc oxide binds to phosphate (PO₄³⁺) salts found in the gastrointestinal track. For pharmaceutical uses acceptable sources of zinc oxide are preferably United States Pharmacopeia (USP) grade. For example, zinc oxide is commercially available from Southern Ionics, Inc. located in West Point MS. Excess phosphate (hyperphosphatemia, i.e. a serum phosphorus concentration >5 mg/L inorganic phosphorus level) is associated with renal failure.

Zinc oxide can be administered to a patient in a therapeutic amount sufficient to lower the level of phosphate concentration in serum. Preferably, zinc oxide can be administered to a patient in an amount at least about 0.05 gram per kilogram of body weight per day, more preferably at least about 0.1 gram per kilogram of body weight per day, and still more preferably at least about 0.2 gram per kilogram of body weight per day. The amount of zinc oxide administered to the patient may be less than the amount that will induce hypophosphatemia or other health risks. Accordingly, in some cases, no more than about 5 gram of zinc oxide per body weight per day may be administered to the patient, more preferably less than about 3 gram of zinc oxide is provided to the patient per kilogram body weight per day.

The pharmaceutical composition may optionally contain less than 60 wt % filler, or less than 40 wt % filler, or less than 20 wt % filler, or less than 10 wt % filler, or less than 1 wt % filler. The short term “wt” is an abbreviation of weight.

The pharmaceutical composition may comprise one or more other oral sorbents. These may have the function to remove additional toxins from either serum and/or the gut.

The pharmaceutical composition may comprise an agent to enhance the intestine permeability. The intestinal tissue permeability-enhancing agent may be combined with the carbon agent, hydroxy-loaded zirconium oxide, and/or zinc oxide or replace either or both of these agents in a pharmaceutical composition for use in the present disclosure. In the preferred embodiments, the intestinal tissue permeability-enhancing agent may be selected to include a non-absorbing agent such as ethanol, polyethylene glycol, glycerin, propylene glycol, acetone, and polyvinyl alcohol and mixtures of these agents. The intestinal tissue permeability-enhancing agent may be selected to be substantially non absorbable by the intestine. Advantageously, administration of the intestinal tissue permeability-enhancing agent may minimize absorption by the stomach. In one embodiment, such an agent may be a non absorbable alcohol. The intestinal tissue permeability-enhancing agent may be administered to the patient, similarly to the carbon agent, through a variety of administration routes, including orally, rectally, and through ostomy inlet. Examples of intestinal tissue permeability enhancing agents are described in Koszuta, J.; Carter, J. M.; and S. R. Ash “Effect of Ethanol Perfusion on Creatinine Removal in a Roux-Y Intestinal Segment”, Int'l. J. Artif Organs 14(7): 417-423, 1979, which is incorporated by reference in its entirety.

The pharmaceutical composition may comprise one or more of a water-absorber. For example, it may include a hydrogel. In some embodiments, the water-absorber may be a water absorbing polymer such as, for example, sodium polyacrylate.

The pharmaceutical composition may also include any other medication, for example, a diuretic. Suitable diuretics may include high ceiling/loop diuretics such as furosemide, ethacrynic acid and torasemide; thiazide-type diuretics such as hydrochlorothiazide; carbonic anhydrase inhibitors such as acetazolamide and methazolamide; potassium-sparing diuretics such as aldosterone antagonists and or epithelial sodium channel blockers.

The pharmaceutical composition may include a coating. Advantageously, such coatings may facilitate delivery of the oral sorbent to the colon. The crystalline zirconium phosphate can be administered in combination with a coating encapsulating the crystalline zirconium phosphate.

A particularly convenient approach to a controlled release composition involves encapsulating the crystalline zirconium phosphate with a coating that is resistant to dissociation in the stomach but which is adapted towards dissociation in the small intestine, preferably in the colon. The preparation of a pharmaceutical composition adapted to resist dissociation under acidic conditions but adapted towards dissociation under non-acidic conditions may involve the use of enteric coating. Thereby tablets, capsules, etc., or the individual particles or granules contained therein are encapsulated/coated with a suitable material.

One particular method of encapsulation may involve the use of gelatine capsules coated with a cellulose acetate phthalate diethylphthalate layer. Advantageously, this coating may protect the gelatin capsule from the action of water under the acid conditions of the stomach where the coating is protonated and therefore stable. The coating may be, however, destabilised under the neutral/alkaline conditions of the intestine where it is not protonated, thereby allowing water to act on the gelatin. Other examples of methods of formulation which may be used include the use of polymeric hydrogel formulations which do not actually encapsulate the sorbent but which are resistant to dissociation under acidic conditions. In a preferred embodiment, the coating may be an enteric coating, for example, amyloses or polymethacrylates with a pH-dependent dissolution, for example, eudragit.

The carbon agent can be selected from a wide variety of commercially available pharmaceutically acceptable carbon sources and includes activated carbon, activated charcoal agent, and charcoal sorbent. For example an activated carbon agent can be obtained in USP grade from Mallinckrodt, Inc. of St. Louis, Mo. and can be used in the present disclosure. The carbon agent can be administered to the patient in a therapeutic amount effect to lower the concentration of one or more toxins.

In preferred embodiments, a single unit dose of carbon agent that can provide a therapeutic effect to the patient can be selected to be at least about 0.1 gram of carbon per kg body weight per day, more preferably, at least about 0.2 gram of carbon per kg body weight per day, and still yet more preferably, at least about 0.3 gram of carbon per kg body weight per day. The amount of carbon should not exceed the amount that will promote constipation and/or create a blockage in the patient's intestine. Consequently, it is preferred to include less than about 3 gram of carbon per kg body weight, more preferably, less than about 1.5 gram of carbon per kg body weight per day.

The carbon agent can be administered to the patient through a variety of routes. For example, the carbon agent may be administered orally, rectally, or through an ostomy inlet. The particular pharmaceutical formulation of the carbon agent may vary depending upon the mode of administration, the patient's history and the disease etiology. The carbon agent may be capsulated within a coating such as a cellulose or polymeric coating discussed below in more detail. Alternatively, the carbon agent may be entrained within a carrier or diluent to provide a liquid and/or gel suspension that may be administered to the patient. Additionally, the carbon may be combined in a single medicament or a unit dosage form with the crystalline zirconium phosphate.

The carbon agent may bind to and/or otherwise adsorb a wide variety of toxins particularly organic toxins, which are found in detrimentally high serum concentrations often correlated with liver dysfunction or disease. Examples of toxins adsorbable on the carbon agent include, but are not restricted to, creatinine, bile acids, bilirubin, aromatic amino acids, mercaptans, phenols and homocysteine, uric acid and hippurates.

The pharmaceutical composition may also be administered to an animal. The pharmaceutical composition may be administered to the animal in a unit dosage form. The unit dosage form may provide a therapeutically effective amount of the sorbent and optionally the therapeutically effective agent.

The pharmaceutical composition may be formulated to be administered orally, rectally or through an ostomy inlet. Preferably, the pharmaceutical composition may be an oral sorbent, hence, the crystalline zirconium phosphate may be administered orally. The pharmaceutical composition may be provided as a solid, e.g., a powder, a pill or a pellet; a granule, or a liquid, e.g.,, a suspension, a gel, a paste (e.g., yoghurt-like or spread-like), or a thick or viscous liquid. The pharmaceutical composition may be administered alone as a pill, a granule, or as a suppository, or by enema, or combined with food or drink, or a suppository. The pharmaceutical composition can also be provided as a powder that can be suspended in water or in a milkshake or thick soup, or mixed into ice cream or a variety of other additives. Thus in accordance with the present disclosure, one or more of the additives can be combined with food to provide increased patient comfort and desirability to maintain the strict regime. Additionally the crystalline zirconium phosphate can be admixed with food and/or drinks, or even baked within certain foods such as breads and cookies to facilitate administration. Hence, in another aspect, the crystalline zirconium phosphate may be a food additive or supplementary.

The crystalline zirconium phosphate and the one or more therapeutically effective agent or other additives may be combined in a single pharmaceutical formulation. Alternatively, the crystalline zirconium phosphate sorbent and one or more of the additional agents may be provided in separate pharmaceutical formulations, which may be administered separately to the patient either through the same administration route or through a different administration route.

Certain pharmaceutical compositions provide particularly advantageous results for treating patients suffering from renal and/or liver dysfunction, or renal and hepatic insufficiency. Pharmaceutical formulations can be tailored to the patient's disease state, diet, activity level, and to enhance other existing or recommended treatment regiments, particularly, dialysis treatments. Administering one or more pharmaceutical formulation prepared according to the present invention serves to delay or reduce the frequency of dialysis treatments. Additionally and possible more important from a patient's standpoint, the present disclosure can allow a patient to ingest a more “normal diet”—other than taking one or more of the medicaments—and still significantly reduce the patient's toxin levels. The medicament can be specifically formulated to correspond the patient's diet. This can facilitate better patient compliance with required treatment/medications and contribute to the patient's overall mental state and physical health.

One or more of the additives of the present invention can be provided in tablet, pill, or capsule form all of which can include one or more binders, lubricants, granules and/or coatings. Specific examples of binders for use in the present invention include pharmaceutically accepted binders, such as cellulose and polyethylene glycol (PEG), gum tragacanth, acacia, cornstarch, or gelatin; potato starch, alginic acid and the like; a lubricant, such as magnesium stearate and the like.

Preferably, a coating (and/or encapsulation) provides an enhanced route of administration and greater effectiveness. Preferred pharmaceutical compositions for the ease of preparation and administration includes solid compositions, particularly tablets that are hard-filled or liquid-filled capsules. It is preferred that the coating provides means for absorbing the serum toxins in the intestines, and particularly in the colon, and preferably selectively in the colon. This can include coatings that resist exposing the encapsulated additive from bodily fluids found in the stomach and from the harsh conditions such as the high acidity of the stomach. Preferably the additives are coated with a coating that does not dissolve in the stomach, but does dissolve or release the encapsulated sorbent/additive into the more basic environment found it the intestines and colon, preferably selectively in the colon. Advantageously, a pharmaceutical composition wherein the crystalline zirconium phosphate is coated with an enteric coating, or encapsulated therein, is beneficial for the coating to dissolve selectively in the small intestine and/or in particular the colon, thereby allowing the crystalline zirconium phosphate to absorb the serum toxins selectively in the small intestine and/or in particular the colon.

One or more of the components of the present disclosure can also be combined with a selected carrier, diluent, and/or adjuvant. The carriers are preferably pharmaceutically acceptable carriers that are commercially available. Examples of carriers include starch, lactose, di-calcium phosphate, microcrystalline cellulose, sucrose and kaolin. Liquid carriers include sterile water, polyethylene glycols, non-ionic surfactants, and edible oils such as corn, peanut and sesame seed oils. Adjuvants customarily employed for the preparation of pharmaceutical compositions may advantageously be included, such as flavoring agents sucrose, lactose or saccharin peppermint, oil of wintergreen, or cherry flavoring, coloring agents, preserving agents and anti-oxidants, for example, Vitamin E, BHT and BHA. Dispersions can also be prepared in glycerol, liquid polyethylene glycols and mixtures of oils.

In another aspect, there is provided use of a crystalline zirconium phosphate in the manufacture of a pharmaceutical composition for the treatment of a patient with abnormally high levels of one or more toxins. The pharmaceutical composition may be defined as described for the second aspect herein before.

A patient with abnormally high levels of one or more toxins may be a patient suffering from hyperkalemia, hypercalcemia, hypermagnesemia, hypernatremia, hyperammonemia, hyperphosphatemia, uremia, or complications thereof. “Abnormally high levels of one or more toxins”, as referred to herein, means that the toxin levels of one or more toxins is sufficiently high for the patient to be classified (e.g. to be diagnosed) as having an intoxication and/or a disease associated with such high levels of toxins, for example, as defined by the National Kidney Foundation in the KDIGO 2012, Clinical Practice Guideline for the Evaluation and Management of Chronic Kidney Disease, Official Journal of the International Society of Nephrology, vol. 3, issue 1, January 2013, the content of which is incorporated herein by reference in its entirety.

In particular, the present disclosure may relate to the treatment of patients exhibiting elevated levels of serum toxins. Patients exhibiting elevated levels of serum toxins may have a disease or disorder selected from the group consisting of renal and hepatic insufficiency, in particular selected from a liver or kidney dysfunction (or reduced function), drug overdose, drug interaction and/or trauma. The present disclosure may provide particular advantages for patients suffering in the end stages of chronic renal and liver diseases. The elevated levels of serum toxins may include, but are not limited to, ionic toxins such as ammonium, potassium, sodium, calcium, phosphate, as well as organic toxins including, for example, creatinine, urea, bile acids, bilirubin, aromatic amino acids, mercaptans and the like.

By use of the term patient, it is intended to include human as well as other animals particularly, but not restricted to domesticated mammals, for example a dog, a cat, or a horse.

Preferably, the treatment may include administering, or being administered, a crystalline zirconium phosphate either alone or in combination with other therapeutically effective agents.

The crystalline zirconium phosphate can be administered, or be administered, to the patient in a unit dosage through a variety of routes, including orally, rectally, and through an ostomy inlet. The crystalline zirconium phosphate can be provided in a form depending upon the patient's disease stage, mode of administration and the desired beneficial effect to the patient.

The crystalline zirconium phosphate can be administered, or be administered, individually as a single medication such as a pill, a granule, an enema, or as suppository.

In order to treat patients, particularly patients that exhibit kidney or liver failure such as found, for example, in end stage renal disease, a considerable amount of the selective crystalline zirconium phosphate sorbent may need to be ingested or administered. The exact amount can be calculated by first deciding the percentage decrease in the serum level of toxin removal that would be beneficial for the patient. For example, assuming, particularly in end stage renal disease that the clearance of the toxin is near zero from the body, an intestinal absorbent must absorb a portion of the toxin generated every day that equals the desired decrease in serum concentration. From the expected intestinal concentration of this toxin and Langmuir isotherms, the amount required of sorbent to be ingested or administered can be calculated. For various applications the required amount of crystalline zirconium phosphate sorbent is considered to be between about 15 grams to 30 grams daily for hyperkalemia in kidney failure; between about 50 grams to 300 grams daily to decrease urea in kidney failure; and between about 30 grams to 60 grams daily for hyperammonemia for liver failure. Additionally, one or more of the components of the present disclosure can be administered to treat patients having elevated toxins from lithium overdose, excess plasma ammonium due to drugs such as valproate-comprising medication as well as adverse interactions between prescribed medications.

Accordingly, the crystalline zirconium phosphate, in accordance with the present disclosure, can be administered to the patient in a therapeutically amount effective to reduce the level of one or more serum toxins. In preferred examples, the crystalline zirconium phosphate can be administered to the patient in a single unit dosage amount of between 1 gram and about 100 grams, or between 5 grams and about 80 grams, or between 10 grams and about 50 grams, or between 20 grams and about 40 grams. The patient can be given multiple doses per day. Preferably a therapeutically effective amount of the sorbent can be selected to be between about 0.15 grams and about 5.0 grams per kilogram of body weight daily, or between about 0.5 grams and about 3.0 grams per kilogram of body weight daily, or between about 1.0 grams and about 2.0 grams per kilogram of body weight daily.

In another aspect, the present disclosure provides a method of treating a patient, said method comprising: selecting a patient capable of deriving a benefit from lower levels of one or more serum toxins, comprising administering to said patient a crystalline zirconium phosphate. The method may comprise treating a patient with abnormally high levels of one or more toxins. The method may comprise administering to a patient crystalline zirconium phosphate, or a pharmaceutical composition comprising crystalline zirconium phosphate. The pharmaceutical composition may be defined as described above. The method may include treating patients exhibiting elevated levels of serum toxins. The elevated levels of serum toxins can be, but are not required to be, a result of liver or kidney dysfunction (or reduced function), drug overdose, drug interaction and/or trauma.

The crystalline zirconium phosphate sorbent may be administered in a therapeutic amount effective to lower the concentration of one or more toxins. In one embodiment, the therapeutically effective amount of the pharmaceutical composition may be selected to be between about 0.015 grams and about 5.0 grams per kilogram of body weight.

The method of administration may vary depending upon the patient history, disease etiology and patient and/or disease prognosis.

In addition to reducing the concentration of one or more toxins, the pharmaceutical composition of the present disclosure can increase the serum concentration of selected ionic components. Typically the electrolyte balance for patients must be maintained within certain narrow ranges. Deviation either by retaining too much of a particular component or by having too little of that same component can be equally detrimental and even life threatening to the patient. Maintaining a desired electrolyte balance for patients experiencing either renal or liver dysfunction or having a renal and hepatic insufficiency can be particularly difficult.

In the preferred embodiment, the crystalline zirconium phosphate sorbent may function as a cation exchange. As such, it is preferable to pretreat or prepare the crystalline zirconium phosphate sorbent as above described to include cations which can be exchanged and potentially provide a benefit to the patient, as well as, removing undesirable cations to lower serum toxins from the patient. In one embodiment, the crystalline zirconium phosphate sorbent provided according to the present disclosure can provide at least about 5 mg of calcium per kg of body weight, more preferably at least 30 mg of calcium per kg of body weight per dose. The patient can receive as many doses as medically expedient to increase concentration of the selected cation to within acceptable levels. In a preferred embodiment, the crystalline zirconium phosphate sorbent of the present disclosure can increase the concentration of selected cations for example calcium or magnesium while at the same time reducing the concentration of ionic toxins, e.g., sodium, ammonium and/or potassium. Alternatively, or additionally, the crystalline zirconium phosphate sorbent of the present disclosure can selectively release protons and/or hydronium ions.

It is envisaged that patients can ingest an amount of the sorbent (and therapeutic additive) equal to at least 10% by weight of their daily dietary intake and still gain weight and not lose their appetite. Treating a patient with the pharmaceutical composition of the present disclosure may not provide the same or equivalent clearance of toxins as a normal, healthy, functional kidney and/or liver. However, increasing the amount and efficiency of the clearance drugs, i.e. the sorbent and admixed additive of present disclosure, will increase the amount of toxins eliminated from the body. This will ensure better patient compliance and reduce the reliance of dialysis to remove many toxins, which in turn significantly impacts a patient's life.

Such approaches may involve various types of controlled release systems, ranging from one, which may for example be based on a polymer, which simply provides a delayed release of the oral sorbent with time, through a system which is resistant to dissociation under acidic conditions, for example, by the use of buffering, to a system which is biased towards release under conditions such as prevail in the small intestine, for example, a pH sensitive system which is stabilized towards a pH of 1 to 3 such as prevails in the stomach but not one of 7 to 9 such as prevails in the small intestine. One approach may involve a controlled release system, which is stabilized towards a pH of 1 to 3 such as prevails in the stomach but not one of 5.5 to 7 such as prevails in the colon (large intestine).

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

Features described in the context of “methods of treatment” may correspondingly be applicable to aspects concerning “for use in therapy” or “use of a compound in the manufacture of a pharmaceutical composition for the treatment of a patient with abnormally high levels of one or more toxins”.

In the context of various embodiments, the articles “a”, “an” and “the” as used with regard to a feature or element include a reference to one or more of the features or elements.

As used herein, the term “and/or” includes any and all combinations of one or more of the associated listed items.

### EXAMPLES

### Example 1

### Equilibrium Exchange in Simulated Small Intestinal Fluid

The materials tested are:
hydrogen form of amorphous zirconium phosphate (H-Amorph),
1:1 sodium/hydrogen form of amorphous zirconium phosphate (Na/H(1:1) Amorph),
hydrogen form of alpha-crystalline zirconium phosphate (H-Alpha),
1:1 sodium/hydrogen form of alpha-crystalline zirconium phosphate (Na/H(1:1) Alpha),
hydrogen form of gamma-crystalline phosphate (H-Gamma), and
1:1 sodium/hydrogen form of gamma-crystalline phosphate (Na/H(1:1) Gamma).

The materials were tested in competitive exchange studies, using simulated small intestinal fluid, the measured values for which are shown in Table 1.

TABLE 1
Simulated small intestinal fluid

| Component | Target | Measured |
|---|---|---|
| Sodium (Na) (mEq/L) | 138 | 136 |
| Potassium (K) (mEq/L) | 8.5 | 8.2 |
| Calcium (Ca) (mEq/L) | 10 | 9.62 |
| Hydrogen carbonate (HCO₃) | 30 | 22 |
| (mEq/L) | | |
| Ammonium (NH₄) (mEq/L) | 14 | 12.4 |
| Phosphate (mEq/L) | 2.0 | 1.7 |
| Chloride (Cl) (mEq/L) | 140 | 138 |
| pH | 7.5 | 7.53 |

Three different amounts of each material (10, 50 and 100mg) were suspended in 10 milliliter (mL) each of the simulated intestinal fluid and agitated in a shaker-incubator at 38° C. for 8 hours (h). Agitation was then stopped, and the mixtures were allowed to settle for an additional 1 h. About 2 mL of each supernatant was filtered through a 24 millimeter (mm) 0.2 micrometer (μm) PES syringe filter, and the resulting filtrate was analysed in a lab analyser (Radiometer) for Na, K and Ca, and for NH₄⁺ sing a 96-wellplate essay, the results are shown in Table 2.

TABLE 2

| | | H- | Na/H(1:1) | H- | Na/H(1:1) | H- | Na/H(1:1) |
|---|---|---|---|---|---|---|---|
| Analyte | Amount | Amorph | Amorph | Alpha | Alpha | Gamma | Gamma |
| K | 10 | 7.7 | 7.7 | 8.1 | 8.1 | 8.0 | 8.2 |
| (mEq/L) | 50 | 6.1 | 6.1 | 7.9 | 7.6 | 6.8 | 7.9 |
| Pre: 8.2 | 100 | 5.1 | 4.5 | 7.0 | 7.2 | 5.9 | 6.8 |
| Ca | 10 | 4.86 | 4.46 | 3.76 | 4.80 | 9.60 | 5.14 |
| (mEq/L) | 50 | 4.64 | 2.56 | 0.60 | 3.16 | 9.56 | 4.42 |
| Pre: 9.62 | 100 | 4.04 | 0.34 | 0.20 | 1.38 | 9.42 | 3.80 |
| NH₄ | 10 | 9.90 | 9.99 | 10.82 | 10.45 | 10.00 | 10.92 |
| (mEq/L) | 50 | 8.70 | 8.19 | 10.69 | 10.17 | 9.46 | 10.64 |
| Pre: 12.36 | 100 | 8.55 | 6.15 | 10.12 | 9.90 | 9.03 | 10.02 |
| Na | 10 | 139 | 142 | 139 | 140 | 141 | 141 |
| (mEq/L) | 50 | 132 | 146 | 131 | 138 | 132 | 141 |
| Pre: 136 | 100 | 136 | 151 | 136 | 140 | 128 | 144 |

TABLE 3
(milli equivalents per gram (mEq/g))

| | | Na/ | | Na/ | | Na/ |
|---|---|---|---|---|---|---|
| | H- | H(1:1) | H- | H(1:1) | H- | H(1:1) |
| Analyte | Amorph | Amorph | Alpha | Alpha | Gamma | Gamma |
| K | 0.31 | 0.37 | 0.12 | 0.10 | 0.23 | 0.14 |
| (mEq/g) | | | | | | |
| Ca | 0.56 | 0.92 | 0.94 | 0.82 | 0.02 | 0.58 |
| (mEq/g) | | | | | | |
| NH₄ | 0.38 | 0.62 | 0.22 | 0.25 | 0.33 | 0.23 |
| (mEq/g) | | | | | | |
| Na | 0 | −1.5 | 0 | −0.4 | 0.8 | −0.8 |
| (mEq/g) | | release | | release | removal | release |
| K/Ca | 0.55 | 0.40 | 0.13 | 0.12 | 11.5 | 0.24 |
| selectivity | | | | | | |

The K exchange capacity (see Table 3) was approximately similar for all materials. It was highest for amorphous zirconium phosphate (ZrP) (0.31 and 0.37 mEq/g), and lowest for alpha-crystalline ZrP (0.12 and 0.10 mEq/g). Gamma-crystalline ZrP was slightly better than alpha-crystalline (0.23 and 0.14 mEq/g).

Similarly, the ammonium exchange capacity was roughly comparable for all tested materials. Again, amorphous ZrP was best (0.38 and 0.62 mEq/g), followed by gamma-crystalline ZrP (0.33 and 0.23 mEq/g) and alpha-crystalline ZrP (0.22 and 0.25 mEq/g).

The exchange capacity for Ca, however was subject to significant differences within the tested series. It was high for the amorphous and alpha-crystalline materials, and for the sodium/hydrogen form of gamma ZrP (0.56-0.94 mEq/g), but almost negligible for the hydrogen form of gamma ZrP (0.02 mEq/g).

In consequence, hydrogen gamma-crystalline ZrP provided by far the best selectivity for the removal of potassium versus (vs) calcium (11.5), and was about 20-100-fold more selective than the other test materials (0.13-0.55).

The sodium exchange behaviour of the test materials was as expected, with either a neutral balance (H-amorphous zirconium phosphate and H-alpha zirconium phosphate) or a slight Na removal (0.8 mEq/g for H-Gamma zirconium phosphate), vs sodium release for Na/H(1:1)-Amorph, Na/H(1:1)-Alpha and Na/H(1:1)-Gamma (−1.5, −0.4 and −0.8 mEq/g, respectively).

The hydrogen form of gamma-crystalline ZrP thus shows the best properties for K/Ca selectivity and Na removal, with acceptable removal capacities for K and NH₄.

Experiment 2: Equilibrium Exchange in Simulated Colon Fluid

The materials tested are:
hydrogen form of amorphous zirconium phosphate (H-Amorph),
1:1 sodium/hydrogen form of amorphous zirconium phosphate (Na/H(1:1) Amorph),
hydrogen form of alpha-crystalline zirconium phosphate (H-Alpha),
1:1 sodium/hydrogen form of alpha-crystalline zirconium phosphate (Na/H(1:1) Alpha),
hydrogen form of gamma-crystalline phosphate (H-Gamma), and
1:1 sodium/hydrogen form of gamma-crystalline phosphate (Na/H(1:1) Gamma).

The materials were tested in competitive exchange studies, using simulated colon fluid, the measured values for which are shown in Table 4.

TABLE 4
Simulated colon fluid

| Component | Target | Measured |
|---|---|---|
| Na (mEq/L) | 30 | 28 |
| K (mEq/L) | 75 | 71 |
| Ca (mEq/L) | 20 | 18 |
| Acetate (mEq/L) | 30 | n.d. |
| NH₄ (mEq/L) | 14 | 14 |
| Phosphate (mEq/L) | 20 | 14 |
| Cl (mEq/L) | 125 | 124 |
| pH | 6.0 | 5.83 |

Three different amounts of each material (10, 50 and 100mg) were suspended in 10 mL of the simulated colon fluid and agitated in a shaker-incubator at 38° C. for 8h. Agitation was then stopped, and the mixtures were allowed to settle for an additional 1 h. About 2 mL of each supernatant was filtered through a 24 mm 0.2 μm PES syringe filter, and the resulting filtrate was analysed in a lab analyser (Radiometer) for Na, K and Ca, and for NH₄⁺ using a 96-wellplate essay, the results are shown in Table 5.

TABLE 5

| | | H- | Na/H(1:1) | H- | Na/H(1:1) | H- | Na/H(1:1) |
|---|---|---|---|---|---|---|---|
| Analyte | Amount | Amorph | Amorph | Alpha | Alpha | Gamma | Gamma |
| K | 10 | 72.6 | 72.0 | 73.2 | 74.1 | 71.4 | 72.3 |
| (mEq/L) | 50 | 68.1 | 66.9 | 66.9 | 69.0 | 63.0 | 69.9 |
| Pre: 70.8 | 100 | 63.6 | 62.1 | 65.7 | 65.4 | 54.0 | 68.1 |
| Ca | 10 | 12.09 | 10.08 | 11.58 | 10.32 | 11.70 | 10.11 |
| (mEq/L) | 50 | 13.62 | 8.19 | 11.13 | 10.02 | 17.16 | 9.99 |
| Pre: 18.0 | 100 | 11.31 | 6.30 | 8.43 | 9.99 | 17.61 | 9.87 |
| NH₄ | 10 | 14.42 | 14.11 | 14.07 | 14.02 | 14.03 | 14.03 |
| (mEq/L) | 50 | 13.57 | 13.12 | 13.84 | 14.05 | 13.08 | 14.00 |
| Pre: 14.08 | 100 | 12.95 | 12.01 | 13.37 | 14.05 | 12.03 | 13.99 |
| Na | 10 | 25 | 28 | 25 | 28 | 28 | 28 |
| (mEq/L) | 50 | 28 | 43 | 28 | 31 | 28 | 31 |
| Pre: 28 | 100 | 31 | 58 | 31 | 37 | 28 | 37 |

TABLE 6
(milli equivalents per gram (mEq/g))

| | | H- | Na/H(1:1) | H- | Na/H(1:1) | H- | Na/H(1:1) |
|---|---|---|---|---|---|---|---|
| Analyte | Amount | Amorph | Amorph | Alpha | Alpha | Gamma | Gamma |
| K | Removal | 0.72 | 0.87 | 0.51 | 0.54 | 1.68 | 0.27 |
| (mEq/g) | mEq/g | | | | | | |
| Ca | Removal | 0.67 | 1.17 | 0.96 | 0.80 | 0.04 | 0.81 |
| (mEq/g) | mEq/g | | | | | | |
| NH₄ | Removal | 0.11 | 0.21 | 0.07 | 0.00 | 0.21 | 0.01 |
| (mEq/g) | mEq/g | | | | | | |
| Na | Balance | 0.3 | 3.0 | 0.3 | 0.9 | 0.0 | 0.9 |
| (mEq/g) | mEq/g | | | | | | |
| K/Ca | | 1.07 | 0.74 | 0.53 | 0.68 | 42.0 | 0.33 |
| selectivity | | | | | | | |

The potassium (K) exchange capacity (see Table 6) was similar for all materials, except H-Gamma ZrP, which had approximately double the capacity (1.68 mEq/g) of the next best material, Na-Amorph (0.87 mEq/g). In contrast, the capacity of the Na-form of gamma-crystalline ZrP was lowest in the series, at 0.27 mEq/g.

The ammonium exchange capacity was moderate for H-Gamma, H-Alpha, H-Amorph, and Na-Amorph (0.21, 0.07, 0.11 and 0.21 mEq/g, respectively) but very low for Na-Alpha and Na-Gamma (0.00 and 0.01 mEq/g, respectively).

The exchange capacity for Ca, was again subject to significant differences within the tested series. It was high for all tested materials, except for the hydrogen form of gamma ZrP. Thus, while H-Amorph, Na/H(1:1)-Amorph, H-Alpha, Na/H(1:1)-Alpha and Na/H(1:1)-Gamma had capacities of 0.67 — 1.17 mEq/g, the Ca binding capacity of H-Gamma was only 0.04 mEq/g, more than one order of magnitude lower.

In consequence, hydrogen gamma-crystalline ZrP provided again the by far best selectivity for the removal of potassium vs calcium (42.0). This compares to very low selectivities of the other materials (0.33-1.07), some of which even favour the exchange of Ca over that of K.

All test materials, except hydrogen gamma-crystalline ZrP, release sodium. The largest release happens with the Na-loaded materials Na/H(1:1)-Amorph, Na/H(1:1)-Alpha and Na/H(1:1)-gamma-crystalline ZrP. Hydrogen gamma-crystalline ZrP, on the other hand, maintains a neutral sodium balance.

The hydrogen form of gamma-crystalline ZrP thus also shows the best properties for K/Ca selectivity in the conditions of the colon, combined with very high capacity for the removal of K, acceptable capacity for the removal of NH₄, and neutral Na balance.

While the disclosure has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1-18. (canceled)

19. A method of treating a patient, said method comprising:
selecting a patient capable of deriving a benefit from lower levels of one or more serum toxins, comprising orally administering to said patient a crystalline zirconium phosphate.

20. The method of claim 19, wherein the patient is a human.

21. The method of claim 19, wherein the patient is an animal particularly, but not restricted to a domesticated mammal.

22. The method of claim 21, wherein the domesticated mammal is a dog, a cat, or a horse.

23. The method of claim 19, for the treatment of renal and hepatic insufficiency.

24. The method of claim 19, for the treatment of a patient suffering from hyperkalemia, hypercalcemia, hypermagnesemia, hypernatremia, hyperammonemia, hyperphosphatemia, uremia, or combinations thereof.

25. A food additive or supplementary comprising crystalline zirconium phosphate.

26. (canceled)

27. A method of prevention or treatment of renal and/or hepatic insufficiency, wherein crystalline zirconium phosphate is to be administered orally to a patient.

28. The method of claim 27, wherein more than 50wt % of the crystalline zirconium phosphate is present as α-zirconium phosphate.

29. The method of claim 27, wherein more than 50wt % of the crystalline zirconium phosphate is present as γ-zirconium phosphate.

30. The method of claim 27, wherein the crystalline zirconium phosphate is present as a mixture of α-zirconium phosphate and γ-zirconium phosphate.

31. The method of claim 27, wherein the crystalline zirconium phosphate has an average particle size ranging approximately between 1 micrometers and 500 micrometers.

32. The method of claim 27, wherein the prevention or the treatment of renal and/or hepatic insufficiency comprises treatment of a patient with abnormally high levels of one or more toxins.

33. The method of claim 27, wherein the prevention or the treatment of renal and/or hepatic insufficiency comprises prevention or treatment of kidney failure.

34. The method of claim 27, wherein the crystalline zirconium phosphate is comprised in a pharmaceutical composition that optionally comprises one or more therapeutically effective agents for use in the prevention or treatment of renal and/or hepatic insufficiency, wherein the pharmaceutical composition is to be administered orally.

35. The method of claim 27, wherein the crystalline zirconium phosphate is coated with an enteric coating.

36. The method of claim 35, wherein the enteric coating is configured to dissolve in a patient's colon.

37. The method of claim 36, wherein the crystalline zirconium phosphate is configured to absorb the serum toxins in the patient's colon.

38. The method of claim 27, wherein the patient is a human.

39. The method of claim 27, wherein the patient is an animal.
